(19) United States
(12) Patent Application Publication
(10) Pub. No.: US 2024/0363239 A1
(43) Pub. Date: Oct. 31, 2024

(54) Methods for Automatically Providing Guidance to Patient in Real-Time

(71) Applicant: E-LOVU HEALTH, INC., Cupertino, CA (US)

(72) Inventors: Santosh Pandipati, Los Gatos, CA (US); Noel M. Pugh, San Francisco, CA (US); Kamal Gogineni, Cupertino, CA (US)

(21) Appl. No.: 18/307,757

(22) Filed: Apr. 26, 2023

(51) Int. Cl. G16H 40/63

(52) U.S. Cl. CPC G16H 40/63

(57) ABSTRACT

A patient guidance system includes a data acquisition engine configured to receive multiple input data streams. The multiple input data streams include a stream of current medical data for a patient, a stream of current situational data for the patient, and one or more streams of current environmental characterization data relevant to the patient. The stream of current medical data conveys a current health condition of the patient. The patient guidance system also includes an artificial intelligence model configured to automatically generate a recommendation for the patient in real-time based on the multiple input data streams. The patient guidance system also includes an output processor configured to convey the recommendation to the patient. Methods are also disclosed for automatically providing guidance to a patient in real-time through use of the patient guidance system.

### CROSS-REFERENCE TO RELATED APPLICATIONS

This application is related to U.S. patent application Ser. No. 17/850,951, filed on Jun. 27, 2022, the disclosure of which is incorporated herein by reference in its entirety for all purposes.

### BACKGROUND OF THE INVENTION

Healthcare patients that have certain medical and/or physical conditions may benefit from frequent and consistent consultation with their healthcare provider(s). Often, however, it is difficult or not feasible for a healthcare provider to see and counsel all of their registered patients with a desired frequency and consistency due to the overall number of patients and the limited availability of appointment times. Therefore, the interaction between the patient and their healthcare provider may be less than optimal. Moreover, when the patient and their healthcare provider do interface with each other, the exchange of information between the patient and their healthcare provider is often limited due to time constraints and/or other communication difficulties that may be present, such as the patient's inability to remember and/or articulate their health history in the moment. Also, the information that is exchanged between the patient and their healthcare provider during a given in-person visit is often limited to a small subset of information that characterizes the lifestyle and health of the patient since the previous visit. For this reason, the healthcare provider is often required to work with a partial set of data when characterizing the patient's status and providing recommendations to the patient. The above-mentioned difficulties in patient-to-healthcare provider interaction can be particularly challenging for patients with certain health conditions that require special attention and monitoring, such as pregnancy, heart disease, pulmonary disease, asthma, cancer, and various autoimmune diseases, among many others. For this reason, systems and methods are sought to improve patient-to-healthcare provider interaction and information exchange. It is within this context that the present invention arises.

### SUMMARY OF THE INVENTION

A patient guidance system is disclosed. The patient guidance system includes a data acquisition engine that is configured to receive multiple input data streams. The multiple input data streams include a stream of current medical data for a patient, a stream of current situational data for the patient, and one or more streams of current environmental characterization data relevant to the patient. The stream of current medical data conveys a current health condition of the patient. The patient guidance system also includes an artificial intelligence model that is configured to automatically generate a recommendation for the patient in real-time based on the multiple input data streams. The patient guidance system also includes an output processor that is configured to convey the recommendation to the patient.

In some embodiments, the artificial intelligence model is trained based on case data for a population of patients. In some embodiments, the case data for a given patient within the population of patients includes actions taken and corresponding outcomes as a function of time. Also, the case data for the given patient includes one or more of the multiple input data streams for the given patient as a function of time during periods of time that are relevant to the actions taken and corresponding outcomes present in the case data for the given patient.

In some embodiments, the patient guidance system includes a moderator engine that is configured to moderate the artificial intelligence model with regard to automatic generation of the recommendation for the patient. In some embodiments, the data acquisition engine is configured to receive a current profile for the patient that specifies personal preferences of the patient. The moderator engine is configured to ensure that the recommendation for the patient that is conveyed by the output processor is compatible with the current profile for the patient.

In some embodiments, a method is disclosed for automatically providing guidance to a patient in real-time. The method includes receiving multiple input data streams. The multiple input data streams include a stream of current medical data for a patient, a stream of current situational data for the patient, and one or more streams of current environmental characterization data relevant to the patient. The stream of current medical data conveys a current health condition of the patient. The method also includes executing an artificial intelligence model to automatically generate a recommendation for the patient in real-time based on the multiple input data streams. The method also includes conveying the recommendation to the patient.

In some embodiments, the method includes using case data for a population of patients to train the artificial intelligence model. In some embodiments, the case data for a given patient within the population of patients includes actions taken and corresponding outcomes as a function of time. Also, the case data for the given patient includes one or more of the multiple input data streams for the given patient as a function of time during periods of time relevant to the actions taken and corresponding outcomes present in the case data for the given patient.

In some embodiments, the method includes conducting bi-directional communication between the patient guidance system and the patient without human intervention through operation of a natural language processor. In some embodiments, the method includes directing display of a graphical user interface on a computing system of the patient, where the graphical user interface includes a region for displaying the recommendation for the patient in real-time.

In some embodiments, the method includes receiving a current profile for the patient that specifies personal preferences of the patient. In these embodiments, the method includes moderating the artificial intelligence model with regard to automatic generation of the recommendation for the patient to ensure that the recommendation for the patient is compatible with the current profile for the patient. In some embodiments, the method includes providing feedback into the artificial intelligence model, where the feedback based on the moderating.

In some embodiments, the method includes executing the artificial intelligence model to automatically identify a condition or a situation that will adversely impact the patient when left unmitigated. In these embodiments, the recommendation for the patient is generated to suggest an action by the patient that will mitigate the condition or the situation. In some embodiments, the method includes executing the artificial intelligence model to automatically identify an action that will beneficially impact the patient when performed. In these embodiments, the recommendation for the patient is generated to encourage performance of the action by the patient. In some embodiments, the method includes executing the artificial intelligence model to automatically identify information for conveyance to the patient. In these embodiments, the recommendation for the patient is generated to convey the identified information.

### BRIEF DESCRIPTION OF THE DRAWINGS

FIG. 1A shows a system diagram of the PGS (Patient Guidance System) interfaced with multiple input data streams, in accordance with some embodiments.

FIG. 1B shows an example data flow through the PGS, in accordance with some embodiments.

FIGS. 2A, 2B, and 2C show example patient profile settings that can be specified by the patient to the PGS, in accordance with some embodiments.

FIG. 3 shows an example user interface provided by the PGS to the patient, in accordance with some embodiments.

FIG. 4 shows an example route/task recommendation image that is conveyed to the patient by the PGS, in accordance with some embodiments.

FIG. 5 shows an example of the type of information about the status of the patient that may be conveyed in the patient status data region of the user interface, in accordance with some embodiments.

FIG. 6 shows an example of the type of environmental information that may be conveyed in the environmental status data region of the user interface, in accordance with some embodiments.

FIG. 7 shows an example of the type of alert information that may be conveyed in the alert region of the user interface, in accordance with some embodiments.

FIGS. 8A, 8B, 8C, and 8D show an example dialogue that is carried on between the PGS and the patient, by way of the deep learning engine and the natural language processing AI model therein, in accordance with some embodiments.

FIG. 9A shows a flowchart of a method for automatically providing guidance to the patient in real-time, in accordance with some embodiments.

FIG. 9B shows a flowchart of a method that extends the method of FIG. 9A, in accordance with some embodiments.

### DETAILED DESCRIPTION OF THE INVENTION

In the following description, numerous specific details are set forth in order to provide an understanding of the embodiments disclosed herein. It will be apparent, however, to one skilled in the art that the embodiments disclosed herein may be practiced without some or all of these specific details. In other instances, well known process operations have not been described in detail in order not to unnecessarily obscure the disclosed embodiments.

Embodiments are disclosed herein for a patient guidance system (PGS) that implements real-time, dynamic artificial intelligence (AI)-based predictive analytic modeling of cause-and-effect relationships between content within multiple input data streams and the health/well-being of a patient, in order to automatically generate and convey beneficial and timely recommendations to the patient. In some embodiments, the recommendations are provided as a form of personal coaching. In some embodiments, the recommendations are formulated as behavioral suggestions. In some embodiments, the recommendations are defined to improve lifestyle efficiencies for the benefit of the patient.

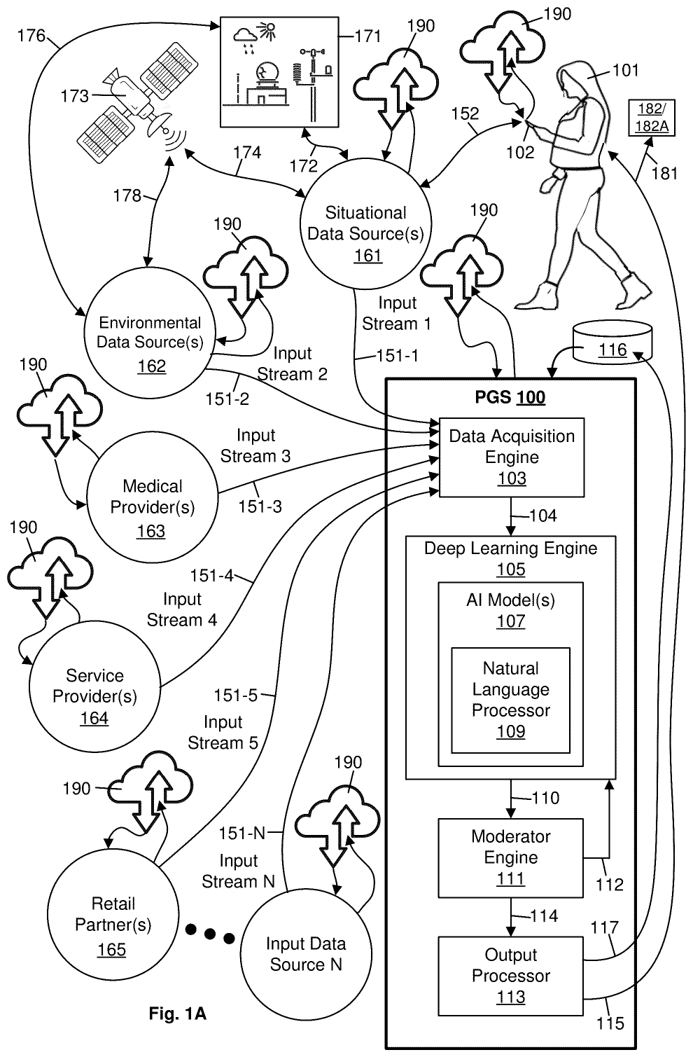

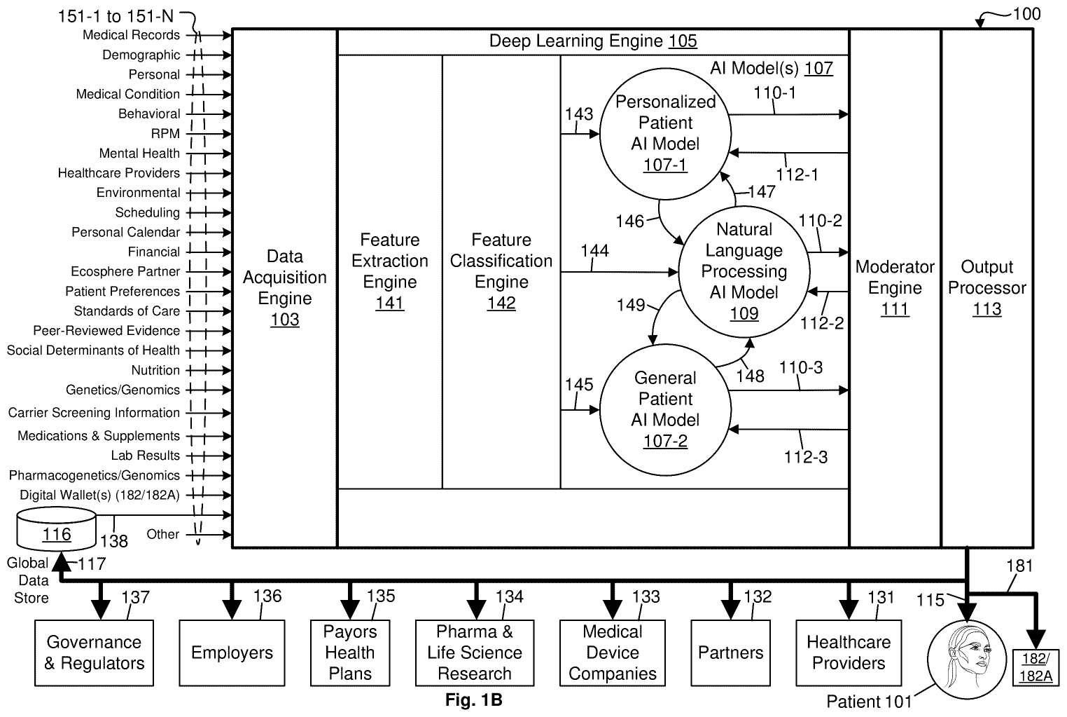

FIG. 1A shows a system diagram of the PGS 100 interfaced with multiple input data streams 151-1 to 151-N, in accordance with some embodiments. The PGS 100 is configured to provide automatic, dynamic, real-time guidance to a patient 101. The PGS 100 includes a data acquisition engine 103 that is configured to take in the multiple input data streams 151-1 to 151-N that are provided/available to the PGS 100. In some embodiments, the data acquisition engine 103 includes a network interface card (NIC) to provide for de-packetization and extraction of data incoming to the PGS 100. The data acquisition engine 103 processes the multiple input data streams 151-1 to 151-N to extract input data and format the input data for provision to a deep learning engine 105, as indicated by arrow 104. The deep learning engine 105 is configured to automatically analyze the data in the various incoming multiple input data streams 151-1 to 151-N in real-time to identify correlations within the data that are indicative of (or potentially indicative of) cause-and-effect relationships that may have a bearing on the health and/or well-being of the patient 101, and/or have a bearing on a health-related outcome associated with the patient 101, e.g., childbirth, chemotherapy, post-procedural recovery, post-operative recovery, etc. In various embodiments, the deep learning engine 105 implements a number of AI model(s) 107 to provide AI-based predictive analysis of cause-and-effect probabilistic correlations that are embedded (and often hidden) within the multiple input data streams 151-1 to 151-N. The AI model(s) 107 function to generate output data that is formulable into beneficial recommendations and/or information for the patient 101. The AI model(s) 107 are trained by a cumulative pool of input data amassed over time from a large population of patients. In some embodiments, health outcomes for the patients in the large population across various sets of health and situational contextual data are used as feedback to train the AI model(s) 107, so that the output data generated by the AI model(s) 107 is directed toward a favorable outcome for the patient 101.

Based on the AI model(s) 107 analysis of the multiple input data streams 151-1 to 151-N, the deep learning engine 105 functions to generate relevant and useful recommendations for the patient 101 in real-time. In some embodiments, the deep leaning engine 105 includes a natural language processor 109 configured to articulate the generated recommendations for the patient 101 in a language that is suitable for the patient 101. In some embodiments, the natural language processor 109 itself is implemented by one or more AI models. In some embodiments, the natural language processor 109 enables dialogue between the PGS 100 (operating autonomously without human involvement) and the patient 101.

The multiple input data streams 151-1 to 151-N include information that is in some way relevant to the patient 101. In various implementations of the PGS 100, the multiple input data streams 151-1 to 151-N can include any number (N) of data streams, where N is an integer greater than one. The example of FIG. 1A shows a first input data stream 151-1 that provides situational data for the patient 101 to the PGS 100. In various embodiments, the situational data for the patient 101 is obtained from one or more situational data source(s) 161. The situational data source(s) 161 include essentially any data source that provides information about a current status of the patient 101. For example, in various embodiments, the situational data source(s) 161 include one or more remote patient monitoring device(s) for providing current medical data for the patient 101, such as blood pressure, heart rate, respiratory rate, body temperature, blood oxygen saturation level, electrocardiogram report, weight, caloric intake, hydration level, glucose level, perspiration level, fetal movement, and fetal heart rate, among others. Also, in various embodiments, the situational data source(s) 161 include one or more activity monitoring source(s) for providing current activity data for the patient 101, such as a global positioning system (GPS) location (e.g., latitude/longitude) of the patient 101 (as obtained from a cellphone of the patient 101), a route of movement/travel of the patient 101 (GPS-based), an exercise/step tracker output for the patient 101, and a sleep/wake detector output for the patient 101, among others. Also, in various embodiments, the situational data source(s) 161 include one or more scheduling data source(s) for providing schedule data for the patient 101, such as an electronic calendar for the patient (e.g., cloud-based calendar, cell phone based calendar, etc.), among others. Also, in various embodiments, the situational data source(s) 161 include one or more communication data source(s) for providing communication data for the patient 101, such as a chat stream, a text message stream, an email communication, a voice communication, a video-captured sign language communication, among others. Also, in various embodiments, the situational data source(s) 161 include one or more subjective data source(s) for providing subjective data for the patient 101, such as a current mood of the patient 101, a current emotion of the patient 101, a current disposition of the patient 101, a current energy level of the patient 101, and a current anxiety level of the patient 101, among others. Also, in various embodiments, the situational data source(s) 161 include one or more financial data source(s) for providing financial data for the patient 101, such as account balances, spending limits, and cost sensitivity metrics, among others. Also, in various embodiments, the situational data source(s) 161 include one or more personal preference settings for the patient 101, such as a daily-life survey for the patient 101 and/or a personal preferences survey of the patient 101.

In various embodiments, one or more of the situational data source(s) 161 can be implemented/enabled by one or more biometric sensors worn by the patient 101 and/or observable of the patient 101. Also, in various embodiments, one or more of the situational data source(s) 161 can be implemented/enabled by an application executing on a personal data communication device 102, e.g., cell phone, of the patient 101, as indicated by arrow 152, where the personal data communication device 102 serves as a communication device to convey data within the first input data stream 151-1 to the PGS 100. Also, in various embodiments, one or more of the situational data source(s) 161 can be implemented/enabled through data communication with a terrestrial-based data communication system 171, as indicated by arrow 172, and/or with a satellite-based data communication system 173, as indicated by arrow 174. Also, in various embodiments, one or more of the situational data source(s) 161 can be in data communication with the PGS 100 through a cloud network 190, e.g., Internet. It should be understood that in various embodiments, the PGS 100 is configured to engage in data communication with essentially any type of communication system and/or network, e.g., cellular, WIFI, satellite, etc.

The example of FIG. 1A shows a second input data stream 151-2 that provides environmental data relevant to the patient 101 to the PGS 100. In various embodiments, the environmental data for the patient 101 is obtained from one or more environmental data source(s) 162. The environmental data source(s) 162 include essentially any data source that provides information about a current status of the environment that may have an impact on the patient 101. For example, in various embodiments, the environmental data source(s) 162 include one or more weather monitoring station(s) for providing current and/or predicted weather data for the region in which the patient 101 is currently located and/or for one or more regions through which the patient 101 is expected/predicted to travel. In various embodiments, the weather data includes a current and/or predicted outdoor temperature, humidity, barometric pressure, precipitation status, precipitation amount, heat index, wind speed, wind direction, and visibility distance, among others. In various embodiments, the environmental data source(s) 162 include one or more air quality monitoring station(s) for providing current and/or predicted air quality data for the region in which the patient 101 is currently located and/or for one or more regions through which the patient 101 is expected/predicted to travel. In various embodiments, the air quality data includes a current and/or predicted air quality index (AQI) value and PM_{2.5} value (a concentration value for particulate matter sized at less than or equal to about 2.5 micrometers), among others.

Also, in various embodiments, the environmental data source(s) 162 includes one or more environmental vector data source(s) for providing data on insect and/or animal activity that impacts vector-borne disease within a region relevant to the patient 101, such as mosquito monitoring data, tick monitoring data, flea monitoring data, bed bug monitoring data, black-fly monitoring data, lice monitoring data, sand-fly monitoring data, triatome bug monitoring data, tsetse-fly monitoring data, aquatic snail monitoring data, and rodent monitoring data, among essentially any other vector monitoring data. Example viruses tracked by the environmental vector data source(s) include one or more of influenza, COVID-19, chikungunya, dengue, Rift Valley fever, yellow fever, Zika, Japanese encephalitis, West Nile fever, phlebotomus fever, Crimean-Congo haemorrhagic fever, and tick-borne encephalitis, among others. Example parasites tracked by the environmental vector data source(s) include one or more of lymphatic filariasis, malaria, schistosomiasis, onchocerciasis, tungiasis, leishmaniasis, Chagas disease, and African trypanosomiasis, among others. Example bacteria tracked by the environmental vector data source(s) include one or more of plague, typhus, Louse-borne relapsing fever, lyme disease, borreliosis, rickettsial diseases, and tularaemia, among others.

In various embodiments, one or more of the environmental data source(s) 162 can be implemented/enabled by an application executing on a personal data communication device 102, e.g., cell phone, of the patient 101, where the personal data communication device 102 serves as a communication device to convey data within the second input data stream 151-2 to the PGS 100. Also, in some embodiments, one or more sensors implemented within the personal data communication device 102 of the patient 101, or connected in data communication with the personal data communication device 102 of the patient 101, are used to measure and report environmental data to the PGS 100. In various embodiments, one or more of the environmental data source(s) 162 can be implemented/enabled through data communication with the terrestrial-based data communication system 171, as indicated by arrow 176, and/or with the satellite-based data communication system 173, as indicated by arrow 178. Also, in various embodiments, one or more of the environmental data source(s) 162 can be in data communication with the PGS 100 through the cloud network 190.

The example of FIG. 1A shows a third input data stream 151-3 that provides medical/healthcare data relevant to the patient 101 to the PGS 100. In various embodiments, the medical/healthcare data for the patient 101 is obtained from one or more medical/healthcare provider(s) 163. The medical/healthcare provider(s) 163 include essentially any entity within the healthcare ecosphere of the patient 101, including medical healthcare provider(s), mental healthcare provider(s), therapy provider(s), general wellness provider(s), specialized wellness provider(s), medical device provider(s), pharmaceutical provider(s), health insurance provider(s), medical regulator(s), medical standards of care provider(s), patient monitoring service provider(s), among essentially any other data source(s) within the healthcare ecosphere of the patient 101. The data provided through the third input data stream 151-3 to the PGS 100 includes the medical history of the patient 101 and the current medical records of the patient 101, as well as specialized reporting from various medical/healthcare provider(s) 163. The current medical condition of the patient 101 is conveyed to the PGS 100 through the third input data stream 151-3. In various embodiments, one or more of the medical/healthcare provider(s) 163 is/are in data communication with the PGS 100 through the cloud network 190.

Also, the example of FIG. 1A shows a fourth input data stream 151-4 that provides information from service provider(s) 164 to the PGS 100. The service provider(s) 164 include any entity outside of the healthcare ecosphere of the patient 101 that provides services to the patient 101. In some embodiments, the service provider(s) 164 include traffic monitoring companies, news providers, delivery companies, cleaning companies, repair service companies, pest control companies, utility companies, phone companies, internet service companies, insurance companies, exercise centers, community centers, massage centers, spas, beauty salons, manicure/pedicure providers, tanning salons, personal shopping services, schools, churches, retreat centers, among essentially any other service provider with which the patient 101 interfaces during their daily-life activities. In various embodiments, one or more of the service provider(s) 164 are in data communication with the PGS 100 through the cloud network 190.

Also, the example of FIG. 1A shows a fifth input data stream 151-5 that provides information from retail partner(s) 165 to the PGS 100. The retail partner(s) 165 include any retail entity outside of the healthcare ecosphere of the patient 101 that offers products for sale to the patient 101. In some embodiments, the retail partner(s) 165 include grocery stores, department stores, restaurants, gas stations, online stores, specialty stores, among essentially any other type of retailer with which the patient 101 interfaces during their daily-life activities. In various embodiments, one or more of the retail partner(s) 165 are in data communication with the PGS 100 through the cloud network 190. In various embodiments, the PGS 100 is configurable to provide automatic, dynamic, real-time interfacing and interaction with any of the service provider(s) 164 and/or retail partner(s) 165 that may be related to the patient 101 in any way, including exchanging data related to particular goods and/or services that are needed by the patient 101, specification of goods and/or services that are available for procurement along with the corresponding prices and times of availability, and transaction processing on behalf of the patient 101.

Essentially any number (N) of data streams can be provided to the PGS 100 from essentially any number of data sources. To illustrate this flexibility, the example of FIG. 1A extends the multiple input data streams 151-1 to 151-N to an N^{th} data stream 151-N provided by an input data source N. Examples of some additional input data sources include an employer of the patient 101, one or more entertainment venues that may be frequented by the patient 101, one or more government offices that may be relevant to an interest of the patient 101 (e.g., office of parks and recreation, etc.), one or more family members of the patient 101, an airline, a hotel, a courier, a ride service, among essentially any other data source that may intersect in some way with the daily-life activity of the patient 101. In some embodiments, the multiple input data streams 151-1 to 151-N include an image and/or video data stream through which images/videos of the body of the patient 101 are conveyed to the PGS 100. In some embodiments, the PGS 100 is configured to receive and process the images/videos of body of the patient 101 as input characterizing the current status of the patient 101. In some embodiments, the PGS 100 is configured to determine differences between images/videos of the body of the patient 101 over time and correlate those differences to the other incoming data in the multiple input data streams 151-1 to 151-N as a function of time in order to identify adverse situations and generate corrective recommendations for the patient 101. In various embodiments, the multiple input data streams 151-1 to 151-N include one or more of medical records data, demographic data, personal data, medical condition data, behavioral data, remote patient monitoring (RPM) data, mental health data, healthcare provider data, environmental data, scheduling data, personal calendar data, financial data, ecosphere partner data, patient preference data, standards of care data, peer-reviewed evidence, social determinants of health data, nutrition data, genetics/genomics data, carrier screening information, medications and supplements information, lab results, pharmacogenetics/genomics, a digital wallet 182 of the patient, among other data.

Also, it should be understood that as time goes on, any of the multiple input data streams 151-2 to 151-N to the PGS 100 may be interrupted temporarily or cancelled. However, the first input data stream 151-1 that conveys current situational data about the patient 101 should continue to be provided to the PGS 100, least the PGS 100 provide output that is not pertinent to a current status of the patient 101. Also, as time goes on, additional input data streams can be conveyed to the data acquisition engine 103 of the PGS 100. In this manner, the overall PGS 100 ecosphere is scalable to adapt to changes in the lifestyle and condition of the patient 101. For example, if the patient 101 were to become immobile, a number of input data streams 151-x, where x is any number from 2 to N, may become irrelevant to the lifestyle of the patient 101. Or, in another example, if the patient 101 were to go on an extended vacation to another country, many of the current input data streams 151-x may become temporarily inactive, while a number of new input data streams 151-y, where y is any number from (N+1) to Y, come online in data communication with the data acquisition engine 103 of the PGS 100. The PGS 100 is configured to automatically adapt to the data that is available in the multiple input data streams 151-1 to 151-N at a given time. For example, if the personal data communication device 102 of the patient 101 is off at a given time so that the PGS 100 is not aware of the exact location of the patient 101 at the given time, the PGS 100 will adapt to operate based on the most applicable data available, such as the most recently obtained forecast environmental data for the region in which the patient 101 is expected to be at the given time. This type of adaptability of the PGS 100 applies to changes in any of the multiple input data streams 151-1 to 151-N over time.

In various embodiments, the multiple input data streams 151-1 to 151-N that are processed by the PGS 100 are obtained from various sources. For example, one or more portable communication devices (e.g., cell phone, laptop, tablet, etc.) associated with the patient 101 can be used to source one or more of the multiple input data streams 151-1 to 151-N. More specifically, one or more applications executing on a personal data communication device 102 of the patient 101 can provide an application programming interface (API) surface area with which the data acquisition engine 103 of the PGS 100 can interact. Also, in some embodiments, one or more applications executing on a personal data communication device 102 of the patient 101 can be configured to regularly convey particular types of data to the PGS 100 by way of the cloud network 190. For example, in various embodiments, the personal data communication device 102 of the patient 101 executes one or more of an exercise/fitness tracking application, a physiological parameter measurement application, a biometric sensor application, a weight tracking application, a mapping application, a GPS application, a calendar application, and a messaging application, among essentially any other type of application, where each executing application provides an API surface area for interaction with the data acquisition engine 103 of the PGS 100. Also, in some embodiments, the PGS 100 interfaces with one or more other data processing/computing systems that have information relative to the patient 101. For example, in some embodiments, the PGS 100 interfaces with one or more of a home security system, a remote monitoring camera system, a home automation system, an automobile, a remote patient monitoring device, a medical device, an in-home air monitoring device, a wearable air monitoring device, an in-home appliance, an environment control system (e.g., thermostat, humidifier, de-humidifier, air filter, etc.), among essentially any other device/system that is associated with the patient 101 and that is capable of data communication with the data acquisition system 103 of the PGS 100. In various embodiments, data communication to/from the PGS 100 is done through the cloud network 190 using any of a number of known network communication protocols. Also, in some embodiments, the PGS 100 is in data communication with the Internet of Things (IoT).

The deep learning engine 105 of the PGS 100 is configured and trained to take in the multiple input data streams 151-1 to 151-N, analyze the multiple input data streams 151-1 to 151-N to identify correlations in real-time that are indicative of cause-and-effect relationships that warrant patient recommendation/communication generation, generate the appropriate patient recommendation/communication, and output the generated patient recommendation/communication for conveyance to the patient 101. In this manner, the PGS 100 continuously synchronizes real-time medical information for the patient 101 with real-time environmental and situational information associated with the patient 101, as obtained from the multiple input data streams 151-1 to 151-N to generate AI-based recommendations for the patient 101 in an automatic, dynamic, and real-time manner, and convey the generated recommendations to the patient 101 in accordance with various communication preferences specified by the patient 101. The AI-based recommendations generated by the PGS 100 for the patient 101 can include essentially any type of recommendation that is actionable by the patient 101 and/or any type of information that is consumable by the patient 101. For example, in various embodiments, the AI-based recommendations generated by the PGS 100 for the patient 101 provide one or more of a recommended course of action by the patient 101, a reminder for the patient 101, a scheduling assist for the patient 101, a statement of advice to the patient 101, and a statement of encouragement for the patient 101, among others. In this manner, the PGS 100 provides an AI-guided future-state for care of the patient 101 that goes well beyond the integration of just the healthcare ecosphere service providers to also include integration of daily-life activities of the patient 101, environmental data associated with the patient 101, business data associated with the patient 101, and essentially any other type of data or data source that intersects with the daily-life and well-being of the patient 101.

In some embodiments, the PGS 100 is configured and trained to provide recommendations/communications to the patient 101 that encourage beneficial patient 101 behavior with regard to physical health and/or mental health, where the beneficial patient 101 behavior may be a behavior already familiar to the patient 101 (and hence input to the PGS 100) and/or a new/different behavior that is automatically and originally generated as a recommendation for the patient 101 by the PGS 100. For example, in some embodiments, PGS 100 correlates data from the multiple input data streams 151-1 to 151-N together to automatically detect a potentially adverse condition and/or situation for the patient 101 and generate a suggested behavioral action for the patient 101 that will mitigate and/or avoid the detected potentially adverse condition and/or situation.

It should be appreciated that the PGS 100 is not simply a data correlation system. Rather, the PGS 100 is a machine learning system that implements AI to consume the multiple input data streams 151-1 to 151-N and creatively and automatically generate output in real-time that is beneficial to the health and well-being of the patient 101, where the output takes the form of recommendations, coaching, and/or information. The PGS 100 serves to automate the processing and correlation of the multiple input data streams 151-1 to 151-N, develop insights into the processed and correlated data, and automatically generate outputs that promote patient 101 engagement and compliance.

With reference back to FIG. 1A, in some embodiments, the output generated by the deep learning engine 105, i.e., the recommendations, coaching, and/or information for the patient 101, is provided as input to a moderator engine 111 before being conveyed outside the PGS 100, as indicated by arrow 110. The moderator engine 111 is configured to operate in either an autonomous mode, a manual mode, or a hybrid mode. In the autonomous mode, the moderator engine 111 operates to automatically ensure that the output generated by the deep learning engine 105 is in compliance with a profile and/or preferences specified by the patient 101, as well as with standards specified by the PGS 100 administrator, who may be a physician of the patient 101 or the physicians designated administrator. In the manual mode, the moderator engine 111 still operates to automatically ensure that the output generated by the deep learning engine 105 is in compliance with the profile and/or preferences specified by the patient 101, as well as with standards specified by the PGS 100 administrator. However, in the manual mode, the moderator engine 111 provides the output generated by the deep learning engine 105 to a moderator portal (e.g., implemented as a graphical user interface) through which the output generated by the deep learning engine 105 can be reviewed and either approved or rejected by a human moderator before it is conveyed outside of the PGS 100 to the patient 101. Also, in the manual mode of operation of the moderator engine 111, the human-moderator decisions on whether or not to allow conveyance of the output generated by the deep learning engine 105 to the patient 101 are fed back into the deep learning engine 105, as indicated by arrow 112, to further train the AI model(s) 107 responsible for generation of the deep learning engine 105 output.

In the hybrid mode, the moderator engine 111 still operates to automatically ensure that the output generated by the deep learning engine 105 is in compliance with the profile and/or preferences specified by the patient 101, as well as with standards specified by the PGS 100 administrator. However, in the hybrid mode, the moderator engine 111 applies a probabilistic confidence assessment to determine a confidence level that the output generated by the deep learning engine 105 is appropriate for conveyance to the patient 101. If the determined confidence level for a given output generated by the deep learning engine 105 meets or exceeds a specified confidence level threshold value, the PGS 100 operates to automatically convey the given output generated by the deep learning engine 105 to the patient 101. However, if the determined confidence level for a given output generated by the deep learning engine 105 is less than the specified confidence level threshold value, the PGS 100 operates to quarantine the given output generated by the deep learning engine 105 for manual moderation, such as through the above-mentioned moderator portal. Then, if the quarantined output of the deep learning engine 105 is reviewed and approved by the human moderator, the quarantined output of the deep learning engine 105 is conveyed from the PGS 100 to the patient 101. Otherwise, the quarantined output of the deep learning engine 105 is discarded within the internal forum of the PGS 100. In some embodiments, the probabilistic confidence assessment to determine the confidence level for the output generated by the deep learning engine 105 with regard to its appropriateness for conveyance to the patient 101 is itself implemented using one or more AI model(s) 107 within the deep learning engine 105.

In some embodiments, the manual mode of operation and the hybrid mode of operation of the moderator engine 111 are used to implement a piloting period during which a physician/clinician gains confidence that the PGS 100 is generating recommendations, coaching, and/or information for the patient 101 that are consistent with what the physician/clinician themselves would provide. Over time, the physician/clinician will gain confidence that the PGS 100 is capable of operating essentially and effectively as a virtual extension of the physician/clinician, albeit with data acquisition and data processing capabilities well-beyond that of a human being. Also, in some embodiments, a panel of experts is used to curate data that is used to train the AI model(s) 107 based on evaluation of outputs generated by the deep learning engine 105. In some embodiments, this curation of data for training the AI model(s) 107 is facilitated by the operating the moderator engine 111 is either the manual mode or the hybrid mode.

In some embodiments, operation of the PGS 100 for a given patient 101 includes receiving as input an initial set of recommendations based on both a care plan established by the given patient's clinician and digital biomarkers, e.g., the given patient's race, the given patient's socioeconomic status, known environmental exposures of the given patient based on place of residence and/or place of employment of the given patient, baseline genetic profile data for the given patient, and baseline vital sign data for the given patient (such as weight, heart rate, blood pressure, temperature, blood oxygen level, and/or any other type of vital sign data), among essentially any other type of biomarker. Also, in some embodiments in which the given patient 101 is a pregnant woman, the PGS 100 is configured to automatically and autonomously generate recommendations for iterative modifications of the care plan for the given patient 101 longitudinally over the timespan of the pregnancy and postpartum, which is output by the PGS 100 and provided to the patient 101 and her clinician, as indicated by arrow 115.

In some embodiments, the output provided by the PGS 100 is stored in the digital wallet 182 for the given patient 101, as indicated by arrow 181. For example, in some embodiments, clinical insights, clinical data, and changes to the longitudinal care plan of the given patient 101 are output by the PGS 100 to the digital wallet 182 of the given patient 101 to provide a clinical record of the course of the patient's pregnancy and postpartum journey. The digital wallet 182 is available for lifelong use by the patient 101. Also, upon birth of the child, a digital wallet 182A is generated for the child as well. In some embodiments, the child's digital wallet 182A includes in utero information. Each of the digital wallets 182 and 182A of the patient 101 and their child is intended to be a living store of health and wellness information, which can continue to be updated and modified by new patient information accrued beyond pregnancy as well as by new medical evidence that provides new insights into patient health status will beyond pregnancy.

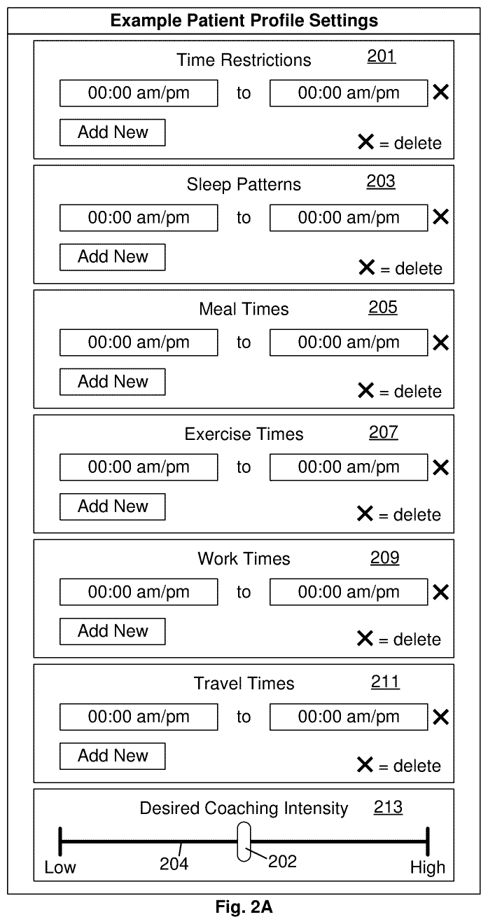

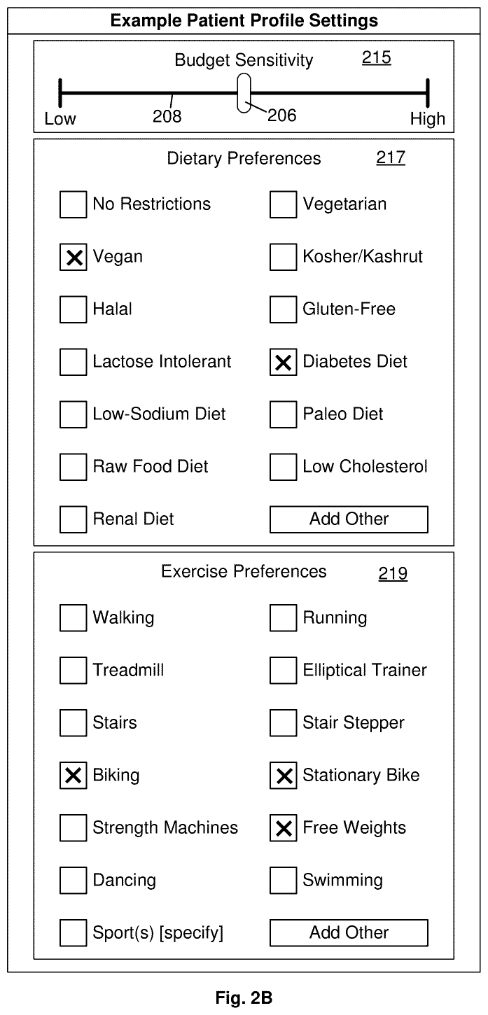

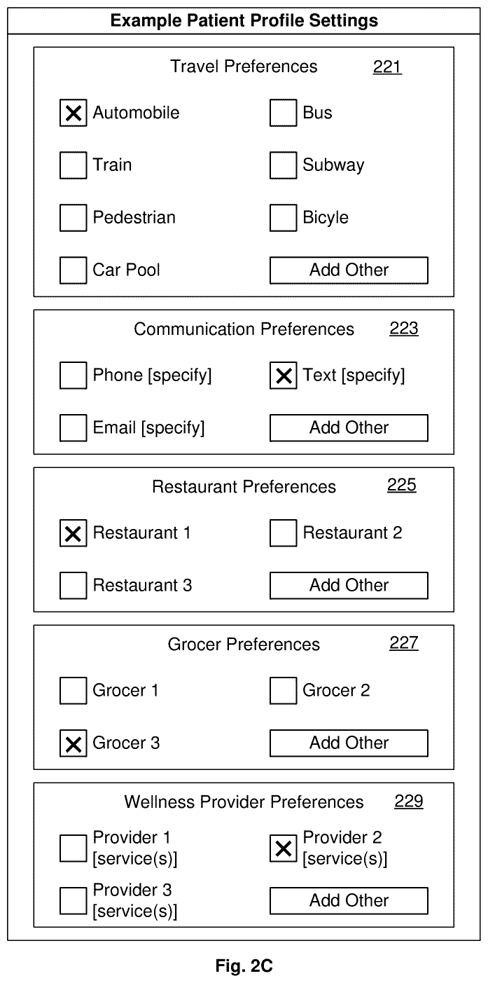

In some embodiments, the PGS 100 is configured to enable the patient 101 to enter patient profile settings for various parameters that establish a baseline of information about the patient 101 and the patient's lifestyle in order to facilitate a low-friction engagement of the patient 101 with the PGS 100. FIGS. 2A-2C show example patient profile settings that can be specified by the patient 101 to the PGS 100, in accordance with some embodiments. In some embodiments, the patient profile settings are initially specified by the patient 101 when setting up a patient account within the PGS 100. In some embodiments, the patient 101 is free to modify their patient profile settings at any time as needed. In various embodiments, the patient profile settings for the PGS 100 establish certain foundational parameters that are used by the moderator engine 111 to control the recommendations, coaching, and/or information that is conveyed to the patient 101 by the PGS 100.

FIG. 2A shows a time restrictions patient profile setting 201 that enables the patient 101 to specify one or more times of the day when the patient 101 considers themselves to be unavailable for communication and/or interaction with the PGS 100. FIG. 2A also shows a sleep patterns patient profile setting 203 that enables the patient 101 to specify one or more times of the day when the patient 101 usually sleeps in order to appropriately control communication and/or interaction by the PGS 100. FIG. 2A also shows a meal times patient profile setting 205 that enables the patient 101 to specify one or more times of the day when the patient 101 usually has meals in order to appropriately control communication and/or interaction by the PGS 100. FIG. 2A also shows an exercise times patient profile setting 207 that enables the patient 101 to specify one or more times of the day when the patient 101 usually does exercise in order to appropriately control communication and/or interaction by the PGS 100. FIG. 2A also shows a work times patient profile setting 209 that enables the patient 101 to specify one or more times of the day when the patient 101 is at work in order to appropriately control communication and/or interaction by the PGS 100. FIG. 2A also shows a travel times patient profile setting 211 that enables the patient 101 to specify one or more times of the day when the patient 101 is usually traveling in order to appropriately control communication and/or interaction by the PGS 100.

FIG. 2A also shows a desired coaching intensity patient profile setting 213 that enables the patient 101 to specify an intensity level at which the patient 101 desired to receive coaching from the PGS 100 in order to appropriately control communication and/or interaction by the PGS 100. In some embodiments, the desired coaching intensity patient profile setting 213 is set by the patient 101 on a continuous or stepped scale extending from a lowest intensity level to a highest intensity level, where the lowest intensity level corresponds to a lowest frequency and/or assertiveness of coaching by the PGS 100, and where the highest level corresponds to a highest frequency and/or assertiveness of coaching by the PGS 100. In the example of FIG. 2A, a control is provided in which the patient 101 is able to click-and-drag a slider 202 along a line 204 to indicate the level of coaching intensity desired by the patient 101.

FIG. 2B is a continuation of the example patient profile settings of FIG. 2A, in accordance with some embodiments. FIG. 2B shows a budget sensitivity patient profile setting 215 that enables the patient 101 to specify a budget sensitivity level that is usable by the PGS 100 when generating recommendations that include expenditure of money by the patient 101. In some embodiments, the budget sensitivity patient profile setting 215 is set by the patient 101 on a continuous or stepped scale extending from a lowest sensitivity level to a highest sensitivity level, where the lowest sensitivity level corresponds to a lowest level of concern about expenses on the part of the patient 101, and where the highest level corresponds to a highest level of concern about expenses on the part of the patient 101. The PGS 100 is configured to use the budget sensitivity patient profile setting 215 as an input when generating recommendations that involve the patient 101 purchasing goods and/or services. The moderator engine 111 also uses the budget sensitivity patient profile setting 215 to ensure that inappropriate spending recommendations are not conveyed to the patient 101 by the PGS 100, especially where such inappropriate spending recommendations could aggravate an anxiety level of the patient 101. For example, if the budget sensitivity patient profile setting 215 is high and/or if the financial information for the patient 101 in the corresponding one of the multiple input data streams 151-1 to 151-N indicates that the patient is in a financially challenged category, then the PGS 100 will take financial status information into consideration so that expensive and/or financially impractical recommendations are not provided to the patient 101 by the PGS 100 and/or so that cost-conscious recommendations are provided to the patient 101 by the PGS 100. In the example of FIG. 2B, a control is provided in which the patient 101 is able to click-and-drag a slider 206 along a line 208 to indicate the budget sensitivity level desired by the patient 101.

FIG. 2B also shows a dietary preferences patient profile setting 217 that enables the patient 101 to specify one or more dietary preferences in order to control and/or moderate food and beverage recommendations generated by the PGS 100. The example dietary preferences patient profile setting 217 enables the patient 101 to select any number of common dietary preferences by selecting check boxes, and optionally by adding new dietary preferences as needed. FIG. 2B also shows an exercise preferences patient profile setting 219 that enables the patient 101 to specify one or more exercise preferences in order to control and/or moderate exercise recommendations generated by the PGS 100. The example exercise preferences patient profile setting 219 enables the patient 101 to select any number of common exercise preferences by selecting check boxes, and optionally by adding new exercise preferences as needed.

FIG. 2C is a continuation of the example patient profile settings of FIGS. 2A and 2B, in accordance with some embodiments. FIG. 2C shows a travel preferences patient profile setting 221 that enables the patient 101 to specify one or more means of travel preferences in order to control and/or moderate recommendations generated by the PGS 100 that involve travel of the patient 101. The example travel preferences patient profile setting 221 enables the patient 101 to select any number of common means of travel preferences by selecting check boxes, and optionally by adding new means of travel preferences as needed. FIG. 2C also shows a communication preferences patient profile setting 223 that enables the patient 101 to specify a preferred manner of communication with the PGS 100 in order to appropriately control communication and/or interaction by the PGS 100. The example communication preferences patient profile setting 223 enables the patient 101 to select any number of common communication preferences by selecting check boxes, and optionally by adding new communication preferences as needed.

FIG. 2C also shows a restaurant preferences patient profile setting 225 that enables the patient 101 to specify one or more restaurant preferences in order to control and/or moderate restaurant recommendations generated by the PGS 100. The example restaurant preferences patient profile setting 225 enables the patient 101 to select any number of restaurant preferences by selecting check boxes, and optionally by adding new restaurant preferences as needed. FIG. 2C also shows a grocer preferences patient profile setting 227 that enables the patient 101 to specify one or more grocery store preferences in order to control and/or moderate grocery store recommendations generated by the PGS 100. The example grocer preferences patient profile setting 227 enables the patient 101 to select any number of grocery store preferences by selecting check boxes, and optionally by adding new grocery store preferences as needed. FIG. 2C also shows a wellness provider preferences patient profile setting 229 that enables the patient 101 to specify one or more wellness provider preferences in order to control and/or moderate wellness provider recommendations generated by the PGS 100. The example wellness provider preferences patient profile setting 229 enables the patient 101 to select any number of wellness provider preferences by selecting check boxes, and optionally by adding new wellness provider preferences as needed. Wellness providers within the context of the wellness provider preferences patient profile setting 229 is essentially any entity that provides goods and/or services that are targeted toward the wellness of the patient 101, including physical therapists, massage therapists, mental health therapists, beauticians, nutritionists, doulas, lactation consultants, personal trainers, and personal shoppers, among essentially any other wellness provider.

With reference back to FIG. 1A, the output of the moderator engine 111 that is to be conveyed to the patient 101 (which represents the output of the deep learning engine 105 that pass the moderation process implemented by the moderator engine 111) is provided to an output processor 113, as indicated by arrow 114. The output processor 113 is configured to format the recommendations, coaching, and/or information for the patient 101, as generated by the PGS 100, for conveyance to the patient 101, as indicated by arrow 115. In some embodiments, the output processor 113 is defined to prepare and transmit the recommendations, coaching, and/or information for the patient 101, as generated by the PGS 100, within data packets over the cloud network 190 to the personal data communication device 102 of the patient 101. In these embodiments, the data packets are prepared by the output processor 113 in accordance with any known and available network communication protocol. In some embodiments, the output processor 113 includes a NIC to provide for packetization of outgoing data to be transmitted from the PGS 100. In some embodiments, the output processor is configured to communicate the output of the PGS 100 and the associated input data to a general data pool 116, as indicated by arrow 117. In some embodiments, the general data pool 116 is maintained within one or more computer readable media in a storage server system of the cloud network 190. However, in various embodiments, the general data pool 116 can be maintained within one or more computer readable media anywhere that is accessible by the PGS 100. Also, in some embodiments, the output processor 113 is configured to communicate information from the PGS 100 to any one or more of the data sources associated with the multiple input data streams 151-1 to 151-N, by way of the cloud network 190. In this manner, the PGS 100 is able to formulate and send instructions and/or data requests directly to any one or more of the data sources associated with the multiple input data streams 151-1 to 151-N.

FIG. 1B shows an example data flow through the PGS 100, in accordance with some embodiments. The various multiple input data streams 151-1 to 151-N are provided as input to the data acquisition engine 103. In some embodiments, the data acquisition engine 103 implements a data filtering system that functions to filter data within the multiple input data streams 151-1 to 151-N to identify specific data relevant to the patient 101. In some embodiments, the data filtering system implements machine learning to identify data specifically tagged/marked for the patient and/or to make predictions about data that may be relevant to the patient 101. In some embodiments, the data acquisition engine 103 implements machine learning to analyze big data that is collected from a population of patients that may have characteristics similar to those of the patient 101. The big data analysis provides for identification of predictive patterns within the data that may be applicable to the patient 101.

After receipt and processing by the data acquisition engine 103, the data from the multiple input data streams 151-1 to 151-N is conveyed to a feature extraction engine 141. The feature extraction engine 141 functions to identify and label features within the data. The identified and labeled features are then conveyed to a feature classification engine 142 that functions to arrange the extracted and labeled features within the data into various classifiers. The classifiers arrange the extracted features in accordance with rules set by the respective classifiers. The feature classification engine 142 then conveys the classified features to the AI model(s) 107. In some embodiments, the feature classification engine 142 conveys the classified features to a personalized patient AI model 107-1, as indicated by arrow 143. In some embodiments, the feature classification engine 142 conveys the classified features to a general patient AI model 107-2, as indicated by arrow 145. Also, in some embodiments, the feature classification engine 142 conveys the classified features to the natural language processing AI model 109, as indicated by arrow 144.

The personalized patient AI model 107-1 builds associations between classified features in order to learn the meaning of the features and relationships between the features. The personalized patient AI model 107-1 is specific to the patient 101. In some embodiments, the deep learning engine 105 generates multiple personalize patient AI models, where each personalized patient AI model is instantiated for a specific patient, to best learn characteristics and identify patterns, trends and insights from the data sources that are relevant to the specific patient. There are various types of machine learning algorithms that can be utilized to form and improve the personalized patient AI model 107-1. In some embodiments, the deep learning engine 105 utilizes methods associated with supervised learning, unsupervised learning, and/or reinforced learning, as known in the art of machine learning (artificial intelligence). In some embodiments, the general patient AI model 107-2 is not specific to any one patient. However, ingestion of larger data sets by the general patient AI model 107-2 may provide more effective AI model training for identifying patterns, trends, and insights within the ingested data. In some embodiments, the personalized patient AI model 107-1 is connected to provide input to the natural language processing AI model 109, as indicated by arrow 146. Also, in some embodiments, the personalized patient AI model 107-1 is connected to receive output from the natural language processing AI model 109, as indicated by arrow 147. In some embodiments, the general patient AI model 107-2 is connected to provide input to the natural language processing AI model 109, as indicated by arrow 148. Also, in some embodiments, the general patient AI model 107-2 is connected to receive output from the natural language processing AI model 109, as indicated by arrow 149.

The personalized patient AI model 107-1 is connected to provide output to the moderator engine 111, as indicated by arrow 110-1. The personalized patient AI model 107-1 is also connected to receive feedback from the moderator engine 111, as indicated by arrow 112-1. The natural language processing AI model 109 is connected to provide output to the moderator engine 111, as indicated by arrow 110-2. The natural language processing AI model 109 is also connected to receive feedback from the moderator engine 111, as indicated by arrow 112-2. The general patient AI model 107-2 is connected to provide output to the moderator engine 111, as indicated by arrow 110-3. The general patient AI model 107-2 is also connected to receive feedback from the moderator engine 111, as indicated by arrow 112-3.

In various embodiments, the output of the PGS 100 is provided to the patient 101, as indicated by arrow 115, and to the general data pool 116, as indicated by arrow 117. Additionally, in various embodiments, the output from the PGS 100 is provided to any of a number of entities within the healthcare ecosphere of the patient 101 and/or external to the healthcare ecosphere of the patient 101. For example, FIG. 1B shows that output from the PGS 100 is provided to healthcare providers 131, partners 132 (e.g., goods and/or services providers), medical device companies 133, pharma and life science research entities 134, health insurance companies 135, employers 136, and government entities 137, among many other possible recipients of output from the PGS 100. Also, in some embodiments, the output from the PGS 100 is provided as feedback data into the PGS 100, as indicated by arrow 138.

The PGS 100 provides more than what would typically be provided by a human-to-human interaction between the patient 101 and their physician/clinician, in that the PGS 100 is able to access and process the multiple input data streams 151-1 to 151-N that have bearing on the health of the patient 101 in real-time in order to automatically determine and communication suggestive beneficial behavior suggestions to the patient 101 in a manner that is not practical or even possible to do by the human physician/clinician. The continuous, automatic, real-time synchronization and correlation of medical data and relevant external data by the PGS 100 in order to provide a machine learning-based determination of the recommended course of action, advice, and/or encouragement for the patient 101 in real-time is a process that extends well beyond the capability of a human physician/clinician. Moreover, through use of the deep learning engine 105, the PGS 100 is able to glean insights for care of the patient 101 that have not been previously observed by the clinical body to date and that would not have been obvious to the clinical body to date. The clinical body as it currently exists does not have the time or human capacity to manually process the vast amounts of diverse data across a large enough population of patients to glean the same insights as can be gleaned by the PGS 100.

In some embodiments, the PGS 100 is applied to provide a type of digital personal coaching model. In these embodiments, the PGS 100 functions as a digital daily coach to help the patient 101 know what they need to do and when to do it, as well as what to avoid and how to avoid it, based on a real-time data acquisition and processing from data sources that intersect with many aspects of the daily-life of the patient 101. The PGS 100 provides a way to shape and improve the behavior of the patient 101 in order to improve the health and well-being of the patient 101. The PGS 100 is seamlessly integrated into the normal environment of the patient 101. In various embodiments, the output of the PGS 100 can be conveyed to the patient 101 in many different ways to improve the ease of consumption and understanding of the PGS 100 output by the patient 101. For example, in some embodiments, recommendation output generated by the PGS 100 for the patient 101 can be provided to the patient 101 as score values and/or as graphical images that convey the information in a quick and meaningful way without overburdening the patient 101 with too much textual reporting and reading.

While the PGS 100 is appropriate for use with any patient having essentially any type of medical diagnosis, the PGS 100 is particularly well-suited for use with a pregnant woman as the patient 101. In this context, the PGS 100 can be applied to effect “personalization” of the pregnancy journey of the mother as the patient 101. Operation of the PGS 100 is seamlessly integrated into the mother's normal environment and daily activities. The PGS 100 implements real-time and dynamic AI-based predictive analytic modeling of cause-and-effect relationships between the content within the multiple input data streams 151-1 to 151-N and the health outcomes of the mother and child in order to automatically generate and convey beneficial behavioral suggestions to the mother. In some embodiments, a goal of implementing the PGS 100 is to actively navigate the mother on a daily basis in accordance with their personal characteristics and behaviors, and based on insights gleaned through application of machine learning to data sets that represent large numbers of patients. In some embodiments, the PGS 100 is used to navigate/guide the patient 101 from pre-conception through two years postpartum. In this manner, the PGS 100 ends up generating an amalgamation of data (a “digital wallet”) for the mother and child from pre-conception through two years postpartum. This digital wallet is beneficial for both the mother and the baby. For example, the information in the digital wallet about the in-utero environment during the pregnancy can be provided to the child's pediatrician to help with medical care for the child.

A woman's ability to come to pregnancy in good health is a strong predictor of the long term health of both the woman and her child. In some embodiments, a pregnancy navigation process includes setting up a personalized care plan at the beginning of the pregnancy, followed by mapping of real-time acquired patient 101 status and activity data against the personalized care plan to see if prescribed goals (e.g., weight gain goals, mental health care appointment goals, nutritional goals, etc.) are being met by the patient 101. If the goals are not being met, the PGS 100 can be used to micronudge the patient 101 in a direction that will help them meet the goals of the personalized care plan. In this manner, the PGS 100 provides support for the mother in the way of coaching and providing micronudges to affect a better behavior of the mother for a better outcome of her pregnancy. For example, micronudges generated by the PGS 100 for the mother could include recommendations encouraging the mother to exercise, to make the right nutritional decisions, and/or to avoid adverse environmental exposures (e.g., excess heat, air pollution, etc.) that contribute cumulatively over the course of the pregnancy toward a potentially adverse outcome for the mother and/or the baby. The PGS 100 functions as a digital daily coach to help the mother know what she needs to do and what she needs to avoid based on real-time data acquisition and processing. The PGS 100 provides for shaping and improving the behavior of the mother in order to improve the mother's health and the healthy development of the baby, and ultimately improve the outcome for both the mother and baby at birth.

The PGS 100 can also be used to improve the daily life of the pregnant mother. For example, if the tool is providing a meal recommendation to the mother, the tool can detect the mother's proximity to, or future route passing by, a food store to ask the mother if she would like the meal recommendation to be fulfilled by the food store. If the mother says yes, the tool would automatically generate and send an order to the food store to prepare the recommended meal and have it ready for pick up by the mother at the anticipated time when the mother will be arriving at the food store. It should be understood that this is just one of an essentially limitless number of ways in which the PGS 100 can be applied to navigate and improve the daily life of the pregnant mother and/or the baby.

In some embodiments, the PGS 100 functions to proactively identify situations that pose elevated risk to the mother and/or the baby and guide the mother to take action to mitigate the risk posed by the situation. For example, the PGS 100 functions to recognize that the current AQI is 156 (which is in the unhealthy zone) and correspondingly recommends to the mother that she drive to her daily workout and wear an N95 mask. In addition, the PGS 100 recommends to the mother that she avoid various hotspots for air pollution as identified through analysis of air quality data from one or more government entities. Again, it should be understood that this is just one of an essentially limitless number of ways in which the PGS 100 can be applied to navigate and improve the daily life of the pregnant mother and/or the baby. It should be understood that the PGS 100 provides more than what would typically be provided by a human-to-human interaction between the expectant mother and her physician/clinician, in that the PGS 100 is able to access and process many external data streams (multiple input data streams 151-1 to 151-N) that have bearing on the health of the mother and the child in the womb in real-time in order to automatically and dynamically determine and communicate suggestive beneficial behavior suggestions to the mother in real-time in a manner that is not practical or even possible to do by the human physician/clinician.

It should be understood that in various embodiments the PGS 100 can function to surface non-obvious insights into relationships between environmental hazards and health/well-being of the patient 101. For example, the PGS 100 can function to surface insights that are not obvious to the average physician, mother, and/or consumer of healthcare with regard to the detrimental effects that environmental hazards can have on pregnancy and overall health. Also, there is no evidence or body of knowledge to date that considers the effect that one patient 101 service provider has on another patient 101 service provider, i.e., interaction effect. However, through application of the PGS 100, such interaction effects can be identified and addressed as needed. For example, consider that it is known that psychiatry has a two standard deviation of impact on an outcome for the patient 101, and that each of physical therapy and nutrition has a given amount of impact on an outcome for the patient 101. However, what is unknown is the degree to which psychiatry, physical therapy, and nutrition interact with each other to create a net new outcome for the patient 101. It should be appreciated that the PGS 100 as disclosed herein is capable of identifying and characterizing interaction outcomes of multi-variate patient 101 treatment functions that prior to the PGS 100 could not be feasibly identified and characterized. Additionally, the PGS 100 can be leveraged to develop and output digital biomarkers to establish baseline markers. Operation of the PGS 100 generates real world evidence to facilitate prediction, diagnosis, monitoring, and management of patient 101 outcomes.

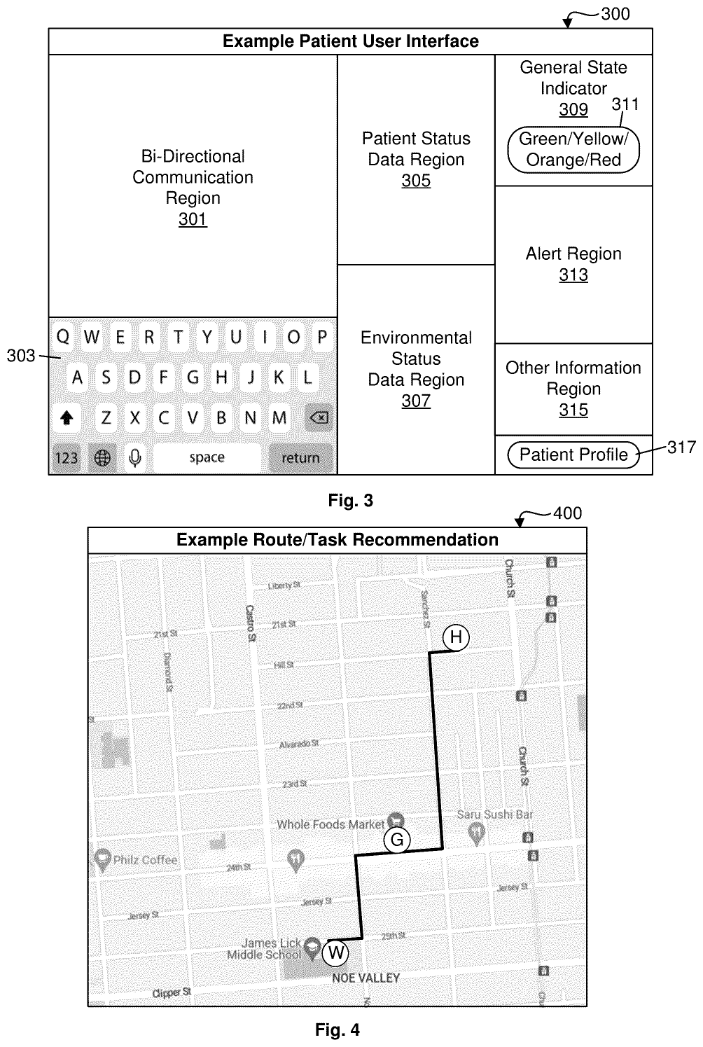

FIG. 3 shows an example user interface 300 provided by the PGS 100 to the patient 101, in accordance with some embodiments. In some embodiments, the personal data communication device 102 of the patient is equipped with a screen on which the user interface 300 is displayed. In some embodiments, the user interface 300 includes a bi-directional communication region 301 in which recommendations, coaching, and/or information are conveyed to the patient 101 by the PGS 100, and through which the patient 101 communicates with the PGS 100. For example, in some embodiments, the bi-directional communication region 301 is implemented as a text messaging region. In these embodiments, a keyboard 303 is surfaced within the user interface 303 to provided for communication by the patient 101 to the PGS 100. In some embodiments, the personal data communication device 102 of the patient is equipped with a microphone through which the patient 101 can audibly communicate with the PGS 100. In these embodiments, the PGS 100 includes functionality to parse and interpret audible communication received from the patient 101. In some embodiments, the personal data communication device 102 of the patient is equipped with a camera through which the patient 101 can visually communicate with the PGS 100, such as by taking pictures and/or video. In these embodiments, the PGS 100 includes functionality to interpret the images/video communication received from the patient 101.

In some embodiments, the bi-directional communication region 301 displays images conveyed to the patient 101 by the PGS 100. For example, FIG. 4 shows an example route/task recommendation image 400 that is conveyed to the patient 101 by the PGS 100, in accordance with some embodiments. In the example of FIG. 4, the PGS 100 recognizes that the patient 101 is currently at work at James Lick Middle School. The PGS 100 also identifies through the various multiple input data streams 151-1 to 151-N that the patient 101 is in need of vitamins. The PGS 100 learns from the daily routine of the patient 101 that the patient 101 normal takes 25^{th} Street to Sanchez Street to get home (H) from work (W). However, the PGS 100 recognizes that another route can be generated to take the patient 101 by Whole Foods Market (G) on the way from work (W) to home (H) in order to replenish the patient's supply of vitamins and simultaneously secure dinner for the patient 101 that satisfies nutritional goals for the patient 101. The PGS 100 automatically and in real-time functions to generate a recommendation for the patient 101 that they take an alternate route home (H) from work (W) that includes a stop at Whole Foods Market (G) to pick up the needed vitamins and get dinner that meets their nutritional goals. The recommendation is conveyed by the PGS 100 to the patient 101 in the bi-directional communication region 301 and includes the image 400 mapping the alternate route. The patient 101 then uses the bi-directional communication region 301 or microphone or camera to respond to recommendation provided by the PGS 100. Then, if the patient 101 accepts the recommendation provided by the PGS 100, the PGS 100 automatically places the order for the vitamins and dinner with the Whole Foods Market (G) for pick up by the patient 101 at a predicted time when the patient 101 will be leaving work (W) based on the learned routine of the patient 101 by the PGS 100. It should be appreciated that the recommendation generation and conveyance example provided by way of FIG. 4 is one of a limitless number of different recommendations that can be generated by the PGS 100 for various patients 101.

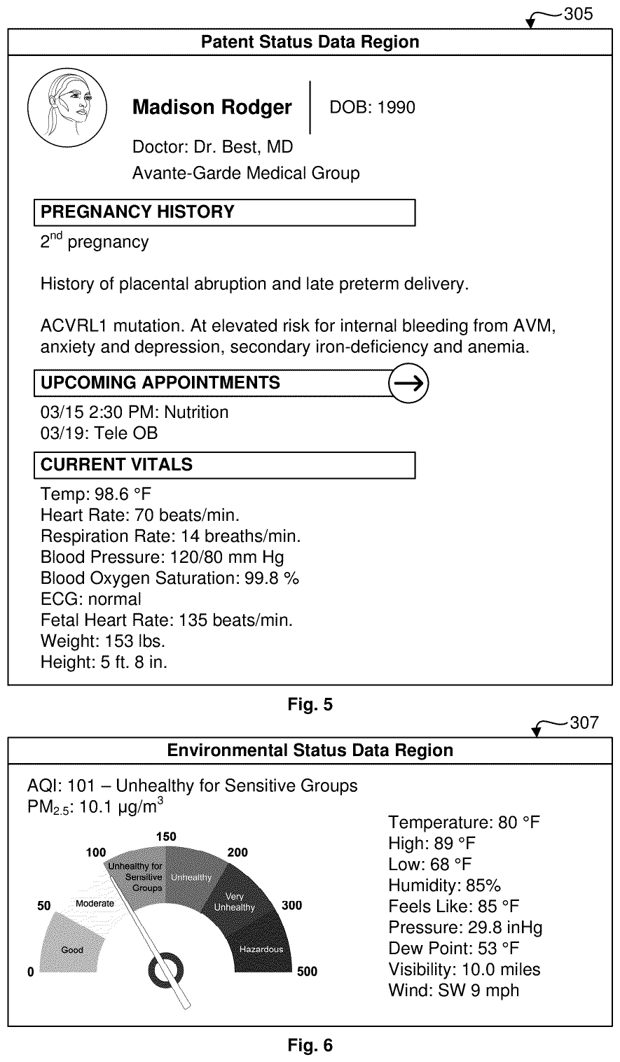

In some embodiments, the user interface 300 also includes a patient status data region 305. FIG. 5 shows an example of the type of information about the status of the patient 101 that may be conveyed in the patient status data region 305, in accordance with some embodiments. It should be understood that the patient 101 information shown in FIG. 5 is provided by way of example and is in no way limiting with regard to the type of information about the status of the patient 101 that may be conveyed in the patient status data region 305. In some embodiments, the user interface 300 also includes an environmental status data region 307. FIG. 6 shows an example of the type of environmental information that may be conveyed in the environmental status data region 307, in accordance with some embodiments. It should be understood that the environment information shown in FIG. 6 is provided by way of example and is in no way limiting with regard to the type of environmental information that may be conveyed in the environmental status data region 305.

In some embodiments, the user interface 300 also includes general state indicator region 309 that includes a graphic 311 that conveys a general state of the health and/or well-being of the patient 101. In some embodiments, the graphic 311 is similar to a light that conveys a certain color that connotes a certain general state of the health and/or well-being of the patient 101. For example, the color green in the graphic 311 may connote that the PGS 100 is determining that all is currently well with the patient 101. The color yellow in the graphic 311 may connote that the PGS 100 is currently determining that something of relatively minor importance should be considered by the patient 101. The color orange in the graphic 311 may connote that the PGS 100 is currently determining that something of relatively significant importance should be considered by the patient 101. The color red in the graphic 311 may connote that the PGS 100 is currently determining that something of urgent significance should be immediately addressed by the patient 101.

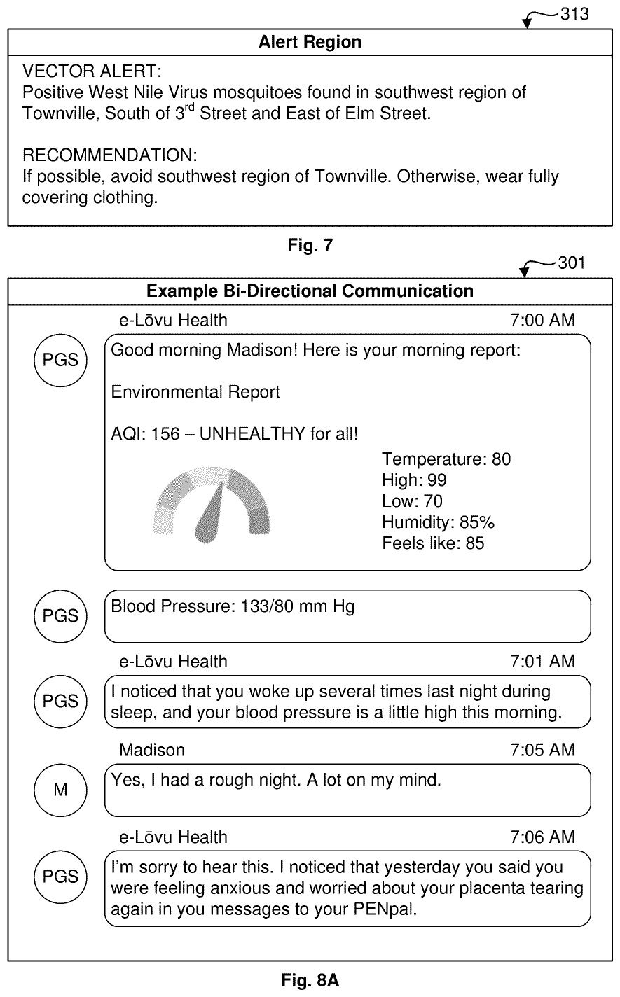

In some embodiments, the user interface 300 also includes an alert region 313 in which the PGS 100 conveys various types of alert information to the patient 101. FIG. 7 shows an example of the type of alert information that may be conveyed in the alert region 313, in accordance with some embodiments. It should be understood that the alert information shown in FIG. 7 is provided by way of example and is in no way limiting with regard to the type of alert information that may be conveyed in the alert region 313. In some embodiments, the user interface 300 also includes an other information region 315 in which the PGS 100 conveys various types of other (e.g., general) information to the patient 101. Also, in some embodiments, the user interface 300 includes a control 317 that when activated by the patient 101 will surface the profile settings for the patient 101 as shown in FIGS. 2A-2C.

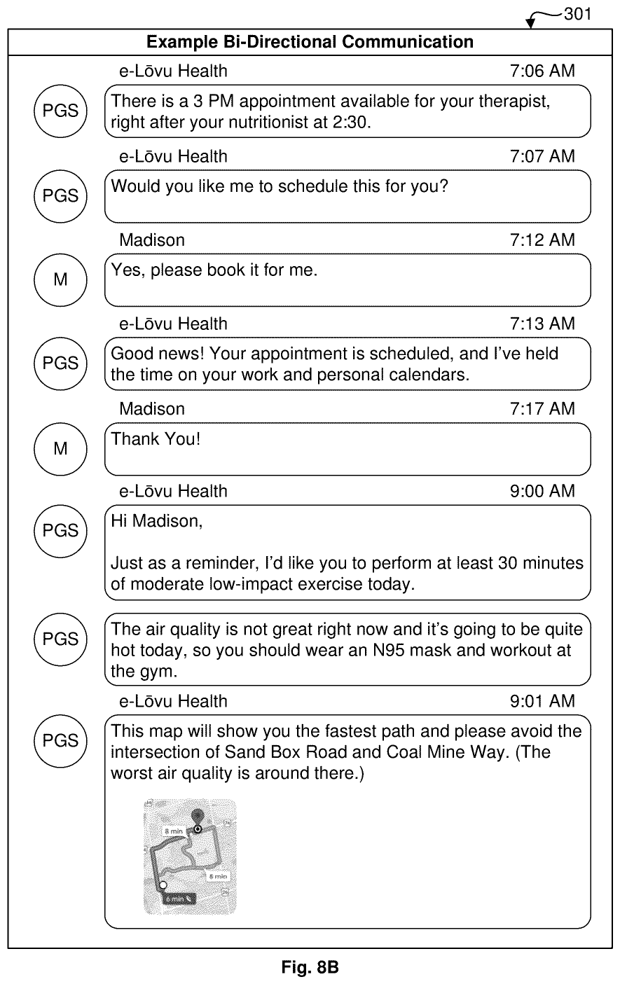

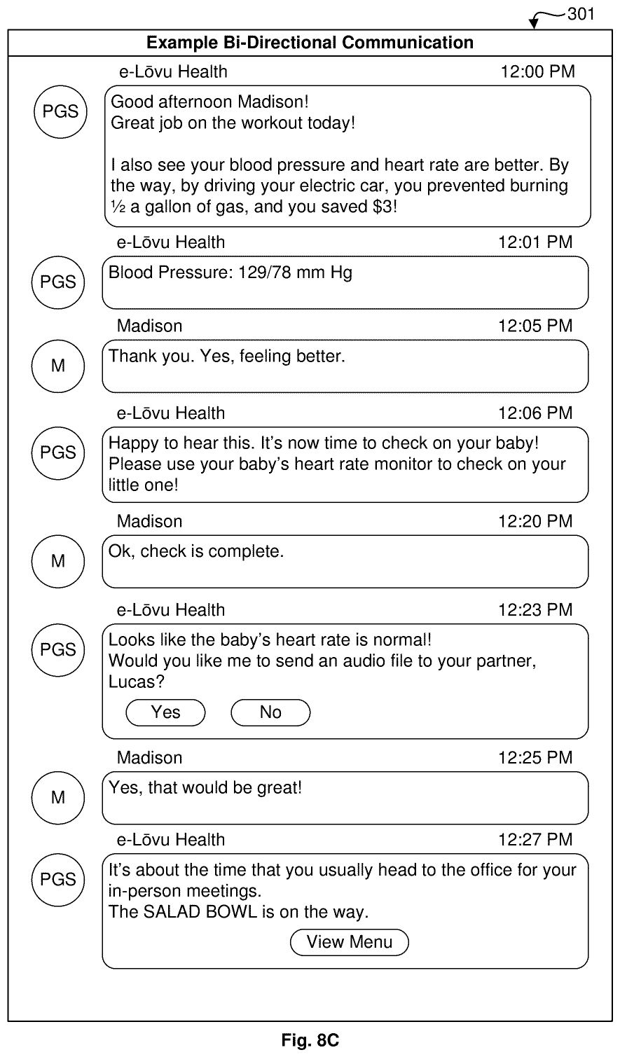

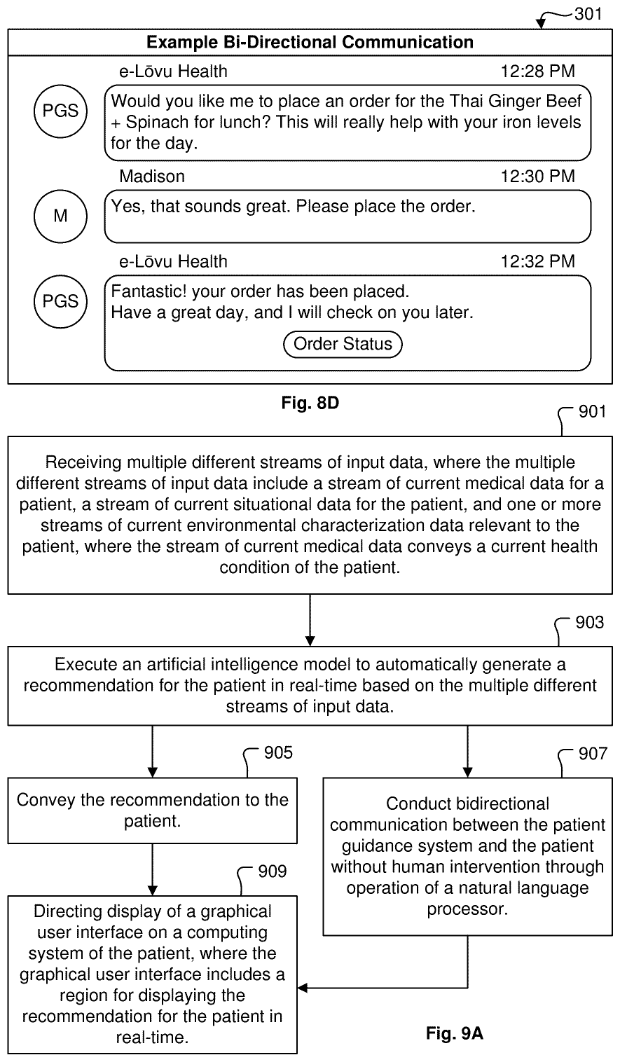

As mentioned with regard to FIGS. 1A and 1B, the deep learning engine 105 includes the natural language processing AI model 109 that is capable of automatically generating conversational statements and phrases to carry on a dialogue with the patient 101 as needed per the operation of the PGS 100. To this end, FIGS. 8A through 8D show an example dialogue that is carried on between the PGS 100 and the patient 101, by way of the deep learning engine 105 and the natural language processing AI model 109 therein, in accordance with some embodiments. The example dialogue of FIGS. 8A through 8D is carried on through the bi-directional communication region 301 of the user interface 300. The phrases/statements/information that is automatically generated and conveyed to the patient 101 by the PGS 100 are denoted by the moniker “PGS.” The responses provided by the patient 101 are denoted by the moniker “M.” It should be understood that the example dialogue shown in FIGS. 8A through 8D is provided by way of example and is in no way limiting with regard to the dialogue that may be carried on between the PGS 100 and the patient 101 by way of the deep learning engine 105 and the natural language processing AI model 109 therein.

In an example embodiment, the PGS 100 includes the data acquisition engine 103, the artificial intelligence model 107, and the output processor 113. The data acquisition engine 103 is configured to receive the multiple input data streams 151-1 to 151-N. In some embodiments, the data acquisition engine 103 is configured for data connection with one or more applications executing on a computing device of the patient 101, e.g., the personal data communication device 102, where the one or more applications provide one or more of the multiple input data streams 151-1 to 151-N to the data acquisition engine 103. The artificial intelligence model 107 is configured to automatically generate a recommendation for the patient 101 in real-time based on the multiple input data streams 151-1 to 151-N. The output processor 113 is configured to convey the recommendation to the patient 101. In some embodiments, the artificial intelligence model 107 is trained based on case data for a population of patients, where the case data for a given patient within the population of patients includes actions taken and corresponding outcomes as a function of time. Also, the case data for the given patient within the population of patients includes one or more of the multiple input data streams 151-1 to 151-N for the given patient as a function of time during periods of time relevant to the actions taken and corresponding outcomes present in the case data for the given patient.

In some embodiments, the artificial intelligence model 107 is configured to automatically identify a condition or a situation that will adversely impact the patient 101 when left unmitigated. In these embodiments, the recommendation for the patient 101 is automatically generated by the PGS 100 to suggest an action by the patient 101 that will mitigate the condition or the situation. In some embodiments, the artificial intelligence model 107 is configured to automatically identify an action that will beneficially impact the patient 101 when performed. In these embodiments, the recommendation for the patient 101 is automatically generated by the PGS 100 to encourage performance of the action by the patient 101. In some embodiments, the artificial intelligence model 107 is configured to automatically identify information for conveyance to the patient 101. In these embodiments, the recommendation for the patient 101 is automatically generated by the PGS 100 to convey the identified information.

The multiple input data streams 151-1 to 151-N include a stream of current medical data for the patient 101, e.g., such as from the medical provider(s) 163. The stream of current medical data conveys a current health condition of the patient 101. In some embodiments, the current health condition of the patient 101 is one or more of a woman trying to conceive, a woman that is currently pregnant, and a woman that is within two years postpartum. In some embodiments, the current medical data for the patient 101 includes one or more of a current body temperature, a current heart rate, a current respiration rate, a current blood pressure, a fetal heart rate, a blood oxygen saturation level, and an electrocardiogram. In some embodiments, the current medical data for the patient 101 includes a current body weight and one or more current body measurements. In some embodiments, the current medical data for the patient 101 includes a current medical diagnosis. In some embodiments, the current medical data for the patient 101 includes a current image of one or more body parts of the patient 101.

The multiple input data streams 151-1 to 151-N also include a stream of current situational data for the patient 101, e.g., such as from the situational data source(s) 161. In some embodiments, the stream of current situational data for the patient 101 includes a current location of the patient 101. In some embodiments, the stream of current situational data for the patient 101 includes a current listing of calendared events for the patient 101. In some embodiments, the stream of current situational data for the patient 101 includes a current daily schedule for the patient 101. In some embodiments, the stream of current situational data for the patient 101 includes an activity currently being performed by the patient 101.

The multiple input data streams 151-1 to 151-N also include one or more streams of current environmental characterization data relevant to the patient 101, e.g., such as from the environmental data source(s) 162. In some embodiments, the one or more streams of current environmental characterization data relevant to the patient 101 include one or more of an outdoor temperature value, a humidity value, a barometric pressure value, an air quality index value, a value for particulate matter sized at less than or equal to about 2.5 micrometers (PM_{2.5} value), a heat index value, a wind speed value, a wind direction, a visibility distance value, and an insect/animal vector distribution. In some embodiments, the one or more streams of current environmental characterization data relevant to the patient 101 include one or more air quality measurements within a current vicinity of the patient 101. In some embodiments, the one or more streams of current environmental characterization data relevant to the patient 101 include one or more air quality measurements along an anticipated travel route of the patient 101.

In some embodiments, the PGS 100 includes the natural language processor 109 that is configured to support bi-directional communication between the PGS 100 and the patient 101 without human intervention. In some embodiments, the recommendation for the patient 101 is articulated by the natural language processor 109. In some embodiments, the natural language processor 109 is implemented by an artificial intelligence model.

In some embodiments, the PGS 100 includes the moderator engine 111 that is configured to moderate the artificial intelligence model 107 with regard to automatic generation of the recommendation for the patient 101. In some embodiments, the moderator engine 113 is connected to provide feedback into the artificial intelligence model 107. In some embodiments, the data acquisition engine 103 is configured to receive a current profile for the patient 101 that specifies personal preferences of the patient 101. In these embodiments, the moderator engine 111 is configured to ensure that the recommendation for the patient 101 that is conveyed by the output processor 113 is compatible with the current profile for the patient 101. In some embodiments, the personal preferences of the patient 101 include one or more of budget sensitivity, time restrictions, sleep patterns, dietary preferences, meal times, exercise preferences, entertainment preferences, working hours, work location, travel preferences, travel times, communication preferences, restaurant preferences, grocer preferences, and wellness provider preferences.

In some embodiments, the PGS 100 includes a graphical user interface, e.g., the user interface 300, configured for display on a computing system of the patient 101. In some embodiments, the graphical user interface includes a region, e.g., the bi-directional communication region 301, for displaying the recommendation for the patient 101 in real-time. In some embodiments, the region of the graphical user interface provides for bi-directional communication between the PGS 100 and the patient 101.

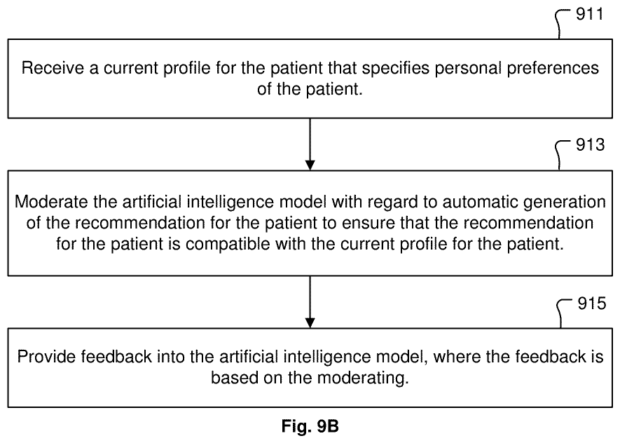

FIG. 9A shows a flowchart of a method for automatically providing guidance to the patient 101 in real-time, in accordance with some embodiments. The method includes an operation 901 for receiving the multiple input data streams 151-1 to 151-N, where the multiple input data streams 151-1 to 151-N include a stream of current medical data for the patient 101, a stream of current situational data for the patient 101, and one or more streams of current environmental characterization data relevant to the patient 101. The stream of current medical data for the patient 101 conveys a current health condition of the patient 101. In some embodiments, one or more of the multiple input data streams 151-1 to 151-N are received from one or more applications executing on a computing device of the patient 101. The method also includes an operation 903 for executing the artificial intelligence model 107 to automatically generate a recommendation for the patient 101 in real-time based on the multiple input data streams 151-1 to 151-N. The method also includes an operation 905 for conveying the recommendation to the patient 101.

In some embodiments, the current health condition of the patient 101 as conveyed in multiple input data streams 151-1 to 151-N in operation 901 is one or more of a woman trying to conceive, a woman that is currently pregnant, and a woman that is within two years postpartum. In some embodiments, the current medical data for the patient 101 as conveyed in multiple input data streams 151-1 to 151-N in operation 901 includes one or more of a current body temperature, a current heart rate, a current respiration rate, a current blood pressure, a fetal heart rate, a blood oxygen saturation level, and an electrocardiogram. In some embodiments, the current medical data for the patient 101 includes a current body weight and one or more current body measurements. In some embodiments, the current medical data for the patient 101 as conveyed in multiple input data streams 151-1 to 151-N in operation 901 includes a current medical diagnosis. In some embodiments, the current medical data for the patient 101 as conveyed in multiple input data streams 151-1 to 151-N in operation 901 includes a current image of one or more body parts of the patient 101.

In some embodiments, the stream of current situational data for the patient 101 as conveyed in multiple input data streams 151-1 to 151-N in operation 901 includes a current location of the patient 101. In some embodiments, the stream of current situational data for the patient 101 as conveyed in multiple input data streams 151-1 to 151-N in operation 901 includes a current listing of calendared events for the patient 101. In some embodiments, the stream of current situational data for the patient 101 as conveyed in multiple input data streams 151-1 to 151-N in operation 901 includes a current daily schedule for the patient 101. In some embodiments, the stream of current situational data for the patient 101 as conveyed in multiple input data streams 151-1 to 151-N in operation 901 includes an activity currently being performed by the patient 101.

In some embodiments, the one or more streams of current environmental characterization data relevant to the patient 101 as conveyed in multiple input data streams 151-1 to 151-N in operation 901 include one or more of an outdoor temperature value, a humidity value, a barometric pressure value, an air quality index value, a value for particulate matter sized at less than or equal to about 2.5 micrometers (PM_{2.5} value), a heat index value, a wind speed value, a wind direction, a visibility distance value, and an insect/animal vector distribution. In some embodiments, the one or more streams of current environmental characterization data relevant to the patient 101 as conveyed in multiple input data streams 151-1 to 151-N in operation 901 include one or more air quality measurements within a current vicinity of the patient 101. In some embodiments, the one or more streams of current environmental characterization data relevant to the patient 101 as conveyed in multiple input data streams 151-1 to 151-N in operation 901 include one or more air quality measurements along an anticipated travel route of the patient 101.

In some embodiments, the operation 903 includes executing the artificial intelligence model 107 to automatically identify a condition or a situation that will adversely impact the patient 101 when left unmitigated. In these embodiments, the recommendation for the patient 101 is generated to suggest an action by the patient 101 that will mitigate the condition or the situation. In some embodiments, the operation 903 includes executing the artificial intelligence model 107 to automatically identify an action that will beneficially impact the patient 101 when performed. In these embodiments, the recommendation for the patient 101 is generated to encourage performance of the action by the patient 101. In some embodiments, the operation 903 includes executing the artificial intelligence model 107 to automatically identify information for conveyance to the patient 101. In these embodiments, the recommendation for the patient 101 is generated to convey the identified information.

In some embodiments, the method further includes using case data for a population of patients to train the artificial intelligence model 107, where the case data for a given patient within the population of patients includes actions taken and corresponding outcomes as a function of time, and where the case data for the given patient also includes one or more of the multiple input data streams 151-1 to 151-N for the given patient as a function of time during periods of time relevant to the actions taken and corresponding outcomes present in the case data for the given patient. In some embodiments, the method also includes an operation 907 for conducting bi-directional communication between the PGS 100 and the patient 101 without human intervention through operation of the natural language processor 109. In some embodiments, the method includes operation of the natural language processor 109 to articulate the recommendation for the patient 101. In some embodiments, the method includes executing an artificial intelligence model to implement the natural language processor 109. In some embodiments, the method also includes an operation 909 for directing display of a graphical user interface on a computing system of the patient 101. In some embodiments, the graphical user interface includes a region for displaying the recommendation for the patient 101 in real-time. In some embodiments, the region within the graphical user interface provides for bi-directional communication between the PGS 100 and the patient 101.

FIG. 9B shows a flowchart of a method that extends the method of FIG. 9A, in accordance with some embodiments. In some embodiments, the method of FIG. 9B is performed in parallel with the method of FIG. 9A. The method includes an operation 911 for receiving a current profile for the patient 101 that specifies personal preferences of the patient 101. The method also includes an operation 913 for moderating the artificial intelligence model 107 with regard to automatic generation of the recommendation for the patient 101 to ensure that the recommendation for the patient 101 that is provided by the PGS 100 is compatible with the current profile and personal preferences of the patient 101. In some embodiments, the method also includes an operation 915 for providing feedback into the artificial intelligence model 107, where the feedback is based on the moderating performed in the operation 913. In some embodiments, the personal preferences of the patient 101 include one or more of budget sensitivity, time restrictions, sleep patterns, dietary preferences, meal times, exercise preferences, entertainment preferences, working hours, work location, travel preferences, travel times, communication preferences, restaurant preferences, grocer preferences, and wellness provider preferences.

Embodiments of the present invention may be practiced with various computer system configurations including servers, cloud systems, hand-held devices, microprocessor systems, microprocessor-based or programmable consumer electronics, minicomputers, mainframe computers and the like. The invention can also be practiced in distributed computing environments where tasks are performed by remote processing devices that are linked through a wire-based or wireless network.

With the above embodiments in mind, it should be understood that the invention could employ various computer-implemented operations involving data stored in computer systems. These operations are those requiring physical manipulation of physical quantities. Usually, though not necessarily, these quantities take the form of electrical or magnetic signals capable of being stored, transferred, combined, compared and otherwise manipulated.

Any of the operations described herein that form part of the invention are useful machine operations. The invention also relates to a device or an apparatus for performing these operations. The apparatus can be specially constructed for the required purpose, or the apparatus can be a general-purpose computer selectively activated or configured by a computer program stored in the computer or storage in cloud systems. In particular, various general-purpose machines can be used with computer programs written in accordance with the teachings herein, or it may be more convenient to construct a more specialized apparatus to perform the required operations.

The invention can also be embodied as computer readable code on a computer readable medium. The computer readable medium is any data storage device that can store data, which can thereafter be read by a computer system. The computer readable medium can also be distributed over a network-coupled computer system so that the computer readable code is stored and executed in a distributed fashion.

The foregoing description of the embodiments has been provided for purposes of illustration and description, and is not intended to be exhaustive or limiting. Individual elements or features of a particular embodiment are generally not limited to that particular embodiment, but, where applicable, are interchangeable and can be used in a selected embodiment, even if not specifically shown or described. In this manner, one or more features from one or more embodiments disclosed herein can be combined with one or more features from one or more other embodiments disclosed herein to form another embodiment that is not explicitly disclosed herein, but rather that is implicitly disclosed herein. This other embodiment may also be varied in many ways. Such embodiment variations are not to be regarded as a departure from the disclosure herein, and all such embodiment variations and modifications are intended to be included within the scope of the disclosure provided herein.

Although some method operations may be described in a specific order herein, it should be understood that other housekeeping operations may be performed in between method operations, and/or method operations may be adjusted so that they occur at slightly different times or simultaneously or may be distributed in a system which allows the occurrence of the processing operations at various intervals associated with the processing, as long as the processing of the method operations are performed in a manner that provides for successful implementation of the method.

Although the foregoing embodiments have been described in some detail for purposes of clarity of understanding, it will be apparent that certain changes and modifications can be practiced within the scope of the appended claims. Accordingly, the embodiments disclosed herein are to be considered as illustrative and not restrictive, and are therefore not to be limited to just the details given herein, but may be modified within the scope and equivalents of the appended claims.

What is claimed is:

1. A method for automatically providing guidance to a patient in real-time, comprising:
receiving multiple input data streams, wherein the multiple input data streams include a stream of current medical data for a patient, a stream of current situational data for the patient, and one or more streams of current environmental characterization data relevant to the patient, wherein the stream of current medical data conveys a current health condition of the patient;
executing an artificial intelligence model to automatically generate a recommendation for the patient in real-time based on the multiple input data streams; and
conveying the recommendation to the patient.

2. The method as recited in claim 1, wherein the current health condition of the patient is one or more of a woman trying to conceive, a woman that is currently pregnant, and a woman that is within two years postpartum.

3. The method as recited in claim 2, wherein the current medical data for the patient includes one or more of a current body temperature, a current heart rate, a current respiration rate, a current blood pressure, a fetal heart rate, a blood oxygen saturation level, and an electrocardiogram.

4. The method as recited in claim 3, wherein the current medical data for the patient includes a current body weight and one or more current body measurements.

5. The method as recited in claim 3, wherein the current medical data for the patient includes a current medical diagnosis.

6. The method as recited in claim 3, wherein the current medical data for the patient includes a current image of one or more body parts.

7. The method as recited in claim 2, wherein the stream of current situational data for the patient includes a current location of the patient.

8. The method as recited in claim 2, wherein the stream of current situational data for the patient includes a current listing of calendared events for the patient.

9. The method as recited in claim 2, wherein the stream of current situational data for the patient includes a current daily schedule for the patient.

10. The method as recited in claim 2, wherein the stream of current situational data for the patient includes an activity currently being performed by the patient.

11. The method as recited in claim 2, wherein the one or more streams of current environmental characterization data includes one or more of an outdoor temperature value, a humidity value, a barometric pressure value, an air quality index value, a value for particulate matter sized at less than or equal to about 2.5 micrometers, a heat index value, a wind speed value, a wind direction, a visibility distance value, and an insect/animal vector distribution.

12. The method as recited in claim 2, wherein the one or more streams of current environmental characterization data includes one or more air quality measurements within a current vicinity of the patient.

13. The method as recited in claim 12, wherein the one or more streams of current environmental characterization data includes one or more air quality measurements along an anticipated travel route of the patient.

14. The method as recited in claim 1, further comprising:
using case data for a population of patients to train the artificial intelligence model, wherein the case data for a given patient within the population of patients includes actions taken and corresponding outcomes as a function of time, the case data for the given patient also including one or more of the multiple input data streams for the given patient as a function of time during periods of time relevant to the actions taken and corresponding outcomes present in the case data for the given patient.

15. The method as recited in claim 1, further comprising:
conducting bi-directional communication between the patient guidance system and the patient without human intervention through operation of a natural language processor.

16. The method as recited in claim 15, wherein the recommendation for the patient is articulated by the natural language processor.

17. The method as recited in claim 15, wherein the natural language processor is implemented by the artificial intelligence model.

18. The method as recited in claim 1, further comprising:
receiving a current profile for the patient that specifies personal preferences of the patient; and
moderating the artificial intelligence model with regard to automatic generation of the recommendation for the patient to ensure that the recommendation for the patient is compatible with the current profile for the patient.

19. The method as recited in claim 18, further comprising:
providing feedback into the artificial intelligence model, the feedback based on the moderating.

20. The method as recited in claim 18, wherein the personal preferences of the patient include one or more of budget sensitivity, time restrictions, sleep patterns, dietary preferences, meal times, exercise preferences, entertainment preferences, working hours, work location, travel preferences, travel times, communication preferences, restaurant preferences, grocer preferences, and wellness provider preferences.

21. The method as recited in claim 1, wherein one or more of the multiple input data streams are received from one or more applications executing on a computing device of the patient.

22. The method as recited in claim 1, further comprising:
directing display of a graphical user interface on a computing system of the patient, the graphical user interface including a region for displaying the recommendation for the patient in real-time.

23. The method as recited in claim 22, wherein the region provides for bi-directional communication between the patient guidance system and the patient.

24. The method as recited in claim 1, further comprising:
executing the artificial intelligence model to automatically identify a condition or a situation that will adversely impact the patient when left unmitigated, wherein the recommendation for the patient is generated to suggest an action by the patient that will mitigate the condition or the situation.

25. The method as recited in claim 1, further comprising:
executing the artificial intelligence model to automatically identify an action that will beneficially impact the patient when performed, wherein the recommendation for the patient is generated to encourage performance of the action by the patient.

26. The method as recited in claim 1, further comprising:
executing the artificial intelligence model to automatically identify information for conveyance to the patient, wherein the recommendation for the patient is generated to convey the identified information.
